(19) United States
(12) Patent Application Publication
(10) Pub. No.: US 2017/0266397 A1
(43) Pub. Date: Sep. 21, 2017

(54) VAPORIZING DEVICE SYSTEM AND METHOD

(71) Applicant: ADVANCED GROW LABS TECHNOLOGIES, LLC, West Haven, CT (US)

(72) Inventors: Chris G. Mayle, Fairfield, CT (US); Stanley Scheufler, Encitas, CA (US)

(73) Assignee: ADVANCED GROW LABS, LLC, West Haven, CT (US)

(21) Appl. No.: 15/465,361

(22) Filed: Mar. 21, 2017

Related U.S. Application Data
(60) Provisional application No. 62/311,146, filed on Mar. 21, 2016.

(51) Int. Cl. A61M 11/04; A61M 15/00; A61M 15/06; A24F 47/00

(52) U.S. Cl. CPC A61M 11/042; A24F 47/00; A61M 15/0021; A61M 15/06; A61M 2205/8206; A61M 2205/3584; A61M 2205/502; A61M 2202/0468; A61M 2205/27; A61M 2202/064; A61M 2205/3331; A61M 2205/3368

(57) ABSTRACT

Embodiments of the disclosure include a system for administering a dosage of an inhalable product to a user including a vaporizing device for converting an active pharmaceutical ingredient (API) into the inhalable product to treat an affliction. The vaporizing device includes a communication element to send/receive at least one piece of dosage data to/from a portable electronic device, the dosage data corresponding to one or more properties of the dosage. The system also includes at least one processor and a memory. Memory includes machine-readable instructions that, when executed by the at least one processor, cause the system to receive an indicator corresponding to the API utilized by the vaporizing device, to determine the dosage for the API utilized by the vaporizing device, to transmit, to the user, instructions for administering the dosage, and to request feedback from the user regarding the efficacy of the dosage.

### CROSS-REFERENCE TO RELATED APPLICATIONS

This application claims the benefit of U.S. Provisional Application No. 62/311,146 filed Mar. 21, 2016, entitled “Vaporizing Device System and Method,” which is incorporated by reference in its entirety.

### BACKGROUND

1. Field of the Invention

The present invention relates to vaporizing devices. More particularly, the present invention relates to systems and methods to administer a therapeutic dose via a vaporizing device.

2. Description of Related Art

Vaporizing devices are utilized to heat an oil or extract (e.g., cannabis oil, tobacco oil, etc.) to generate an inhalable vapor for a user. Instead of igniting the cannabis and/or tobacco to facilitate transmission of the oils to the user, the vaporizing device heats the oils to a temperature that is below combustion, yet enables the active ingredients (e.g., tetrahydrocannabinol (THC), Cannabidiol (CBD), cannabinol (CBN), cannabavarin (THCV), cannabigerol (CBG), cannabichromene (CBC), delta-8-THC, cannabicyclol (CBL), cannabitriol (CBT), and cannabielsoin, etc.) to be converted into a vapor for inhalation and use by the user. Typically, vaporizing devices contain one or more heating elements positioned to transmit energy to the oils or extract to enable the user to receive a dosage of the active ingredients. However, it may be difficult to determine the dosage administered by the vaporizing devices. As a result, inefficiencies arise with the dosing and treatment of a variety of ailments.

### SUMMARY

In an embodiment, a vaporizing device includes a body portion. The body portion includes a heating component positioned at a proximal end of the body portion. The body portion also includes a battery positioned adjacent and electronically coupled to the heating component, the battery providing electrical power to the heating component when the vaporizing device is in a use condition. Additionally, the body portion includes a communication element that transmits at least one piece of dosing information to at least one portable electronic device. The body portion also includes a processor communicatively coupled to the communication element to evaluate the dosing information utilizing machine-readable instructions stored on at least one memory. The vaporizing device also includes a cartridge portion positioned linearly adjacent to the body portion to abut the proximal end. The cartridge portion includes a cartridge body coupled to the body portion and having a chamber to hold an oil for conversion into a vapor by the heating component when the vaporizing device is in the use condition. The cartridge portion also includes a mouthpiece arranged adjacent and fluidly coupled to the cartridge body to enable the vapor to move out of the chamber for inhalation by a user.

In an embodiment, a vaporizing device includes a body portion. The body portion includes a heating component positioned at a proximal end of the body portion. The body portion also includes a battery positioned adjacent and electronically coupled to the heating component, the battery providing electrical power to the heating component when the vaporizing device is in a use condition. Additionally, the body portion includes a communication element that receives at least one piece of dosing information from at least one portable electronic device. Furthermore, the body portion includes a processor communicatively coupled to the communication element to evaluate the dosing information utilizing machine-readable instructions stored on at least one memory. In certain embodiments, the vaporizing device includes a cartridge portion positioned linearly adjacent to the body portion to abut the proximal end. The cartridge portion includes a cartridge body coupled to the body portion and having a chamber to hold an active pharmaceutical ingredient (API). The cartridge portion also includes a mouthpiece arranged adjacent and fluidly coupled to the cartridge body to enable the API to move out of the chamber for inhalation by a user

In an embodiment, a method of administering a dosage with a vaporizing device includes scanning an indicator corresponding to a type of active pharmaceutical ingredient (API) in a cartridge with a personal electronic device, the indicator including cartridge data indicative of a dosage profile for the API in the cartridge. The method also includes transmitting the cartridge data to a server, via the personal electronic device. The method further includes receiving the cartridge data at the server, the server including one or more processors and a memory that stores dosage profiles related to cartridge data. The one or more processors operate to output, to the vaporizing device, a target dosage derived from one or more dosage profiles, the target dosage corresponding to an effective dose for the API corresponding to the cartridge data and one or more ailments suffered by a user. The one or more processors also operate to activate the vaporizing device, by instructing the vaporizing device to activate a heating element, to enable the user to administer the target dosage, the API being converted to vapor via the heating element for inhalation by the user. Furthermore, the one or more processors operate to request feedback from the user, via communication through the personal electronic device, regarding the efficacy of the target dosage.

In another embodiment, a non-transitory computer-readable medium with computer-executable instructions stored thereon executed by one or more processors to perform a method to administer a dosage via a vaporizing device. The method includes receiving an input, from at least one of a personal electronic device or a server, indicative of an active pharmaceutical ingredient (API) disposed within a cartridge for use in treating one or more ailments. The method also includes determining a target dosage based on the input, the target dosage derived from one or more dosage profiles containing the target dosage for one or more ailments corresponding to the input. The method further includes activating the vaporizing device to distribute the target dosage to a user. The method also includes receiving feedback regarding the efficacy of the target dosage for the user.

In a further embodiment, a system for administering a dosage of an inhalable product to a user includes a vaporizing device for converting an oil into the inhalable product. The vaporizing device includes a communication element to send or receive at least one piece of dosage data to or from a portable electronic device, the dosage data corresponding to one or more properties of the target dosage. The system also includes at least one processor and a memory including machine-readable instructions that, when executed by the at least one processor, cause the system to receive an indicator corresponding to the oil utilized by the vaporizing device. The machine-readable instructions, when executed by the at least one processor, also cause the system to determine the target dosage for the oil utilized by the vaporizing device by evaluating the oil and one or more ailments suffered by the user. Moreover, the machine-readable instructions, when executed by the at least one processor, cause the system to transmit, to the user, instructions for administering the target dosage. Additionally, the machine-readable instructions that, when executed by the at least one processor, further cause the system to request feedback from the user regarding the efficacy of the target dosage.

In an embodiment, a method for determining a dosage for an inhalable product includes receiving a first signal indicative of an inhalable product. The method also includes determining a target dosage based on the first signal and a user ailment. The method further includes outputting a feedback request to a user that administered the inhalable product at the target dosage. The method also includes receiving feedback from the user, the feedback corresponding to the efficacy of the target dosage to treat the user ailment. Furthermore, the method includes transmitting the feedback to a server. Additionally, the method includes compiling a plurality of feedback from a plurality of users, each user of the plurality of users having used the inhalable product corresponding to the first signal for the user ailment. The method also includes modifying the target dosage based on the plurality of feedback.

In an embodiment, a cartridge having an oil mixture for use by a vaporizing device includes a first end that couples to the vaporizing device, the first end being proximate to a heating component to transmit heat to the cartridge to vaporize the oil mixture for inhalation. The cartridge also includes a chamber storing the oil mixture to provide a predetermined dose when vaporized and inhaled by a user. The oil mixture includes an active pharmaceutical ingredient (API) utilized to treat one or more physical or psychological ailments, and an excipient added in a quantity proportional to the quantity of API. The oil mixture includes a cannabis oil having a quantity of active tetrahydrocannabinol (THC) and/or Cannabidiol (CBD) or any other active ingredient. The oil mixture also includes polyethylene glycol (PEG) added in a quantity proportional to the quantity of active tetrahydrocannabinol (THC) or CBD or any other active ingredient or combination thereof in the oil such that cartridge containing a cannabis oil having a higher concentration of THC contains a greater amount of PEG than a cartridge containing a cannabis oil having a lower concentration of THC.

In an embodiment, a non-transitory computer-readable medium with computer-executable instructions stored thereon executed by one or more processors to perform a method to administer a dosage via a vaporizing device. The method includes receiving an input, from at least one of a personal electronic device or a server, indicative of an active pharmaceutical ingredient (API) disposed within a cartridge for use in treating one or more ailments. The method also includes determining a target dosage based on the input, the target dosage derived from one or more dosage profiles containing the target dosage for one or more ailments corresponding to the API. The method further includes instructing a user to administer the API at the target dosage. The method also includes receiving feedback regarding the efficacy of the target dosage from the user. The method includes generating a user dosage based on the feedback, the user dosage being different from the target dosage to provide improved efficacy compared to the target dosage.

In an embodiment, a non-transitory computer-readable medium with computer-executable instructions stored thereon executed by one or more processors to perform a method to administer a dosage via a vaporizing device. The method includes receiving an input, via a personal electronic device, indicative of an active pharmaceutical ingredient (API) disposed within a cartridge for use in treating one or more ailments. The method also includes administering a user dosage based on the input, the user dosage corresponding to a user profile containing the user dosage for treating one or more ailments corresponding to the API. The method further includes receiving feedback regarding the efficacy of the user dosage from the user. The method includes modifying the user dosage based on the feedback.

### BRIEF DESCRIPTION OF DRAWINGS

The foregoing aspects, features, and advantages of the present invention will be further appreciated when considered with reference to the following description of embodiments and accompanying drawings. In describing the embodiments of the invention illustrated in the appended drawings, specific terminology will be used for the sake of clarity. However, the invention is not intended to be limited to the specific terms used, and it is to be understood that each specific term includes equivalents that operate in a similar manner to accomplish a similar purpose.

FIG. 1 is a top perspective view of an embodiment of a vaporizing device coupled to a charging element, in accordance with the present disclosure;

FIG. 2 is a block diagram of an embodiment of the vaporizing device of FIG. 1, in accordance with the present disclosure;

FIG. 3 is a schematic side view of an embodiment of a cartridge portion of the vaporizing device of FIG. 1 coupled to a body portion, in accordance with the present disclosure;

FIG. 4 is a top perspective view of an embodiment of a personal electronic device interacting with a cartridge portion of the vaporizing device of FIG. 1, in accordance with the present disclosure;

FIG. 5 is a flow chart of an embodiment of a method for administering a target dosage of an inhalable product, in accordance with the present disclosure;

FIG. 6 is a schematic diagram of an embodiment of a dosage profile, in accordance with the present disclosure;

FIG. 7 is a schematic diagram of an embodiment of a user profile, in accordance with the present disclosure;

FIG. 8 is a schematic diagram of an embodiment of a dosage profile being compared to a user profile, in accordance with the present disclosure;

FIG. 9 is a flow chart of an embodiment of a method for updating a dosage profile, in accordance with the present disclosure;

FIG. 10 is a schematic diagram of an embodiment of a server communicating with personal electronic devices, in accordance with the present disclosure;

FIG. 11 is a flow chart of an embodiment of a method for updating a dosage profile, in accordance with the present disclosure; and

FIG. 12 is a flow chart of an embodiment of a method for administering a target dosage of an inhalable product, in accordance with the present disclosure.

### DETAILED DESCRIPTION

The foregoing aspects, features, and advantages of the present invention will be further appreciated when considered with reference to the following description of embodiments and accompanying drawings. In describing the embodiments of the invention illustrated in the appended drawings, specific terminology will be used for the sake of clarity. However, the invention is not intended to be limited to the specific terms used, and it is to be understood that each specific term includes equivalents that operate in a similar manner to accomplish a similar purpose.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Any examples of operating parameters and/or environmental conditions are not exclusive of other parameters/conditions of the disclosed embodiments. Additionally, it should be understood that references to “one embodiment”, “an embodiment”, “certain embodiments,” or “other embodiments” of the present invention are not intended to be interpreted as excluding the existence of additional embodiments that also incorporate the recited features. Furthermore, reference to terms such as “above,” “below,” “upper”, “lower”, “side”, “front,” “back,” or other terms regarding orientation are made with reference to the illustrated embodiments and are not intended to be limiting or exclude other orientations.

Embodiments of the present disclosure include a vaporizing device for converting an active pharmaceutical ingredient (API) (e.g., oils, extracts, oil/extract mixtures, steroids, anti-inflammatory medications, vaccinations, etc.) containing an active ingredient into a vapor for inhalation by a user. The vaporizing device includes a memory, processor, and communication device to enable tracking of dosing information when the vaporizing device is in use. For example, the vaporizing device may track the duration of an inhalation as the user utilizes the vaporizing device, a dosage amount utilized by the user, the time of day of use, etc. and thereafter transmit the data to a personal electronic device for storage and evaluation. Furthermore, the vaporizing device may receive instructions from the personal electronic device regarding the target dosage, user dosage, inhalation duration, or the like. In this manner, use of the vaporizing device may be monitored and/or controlled. Moreover, a cartridge or packaging containing the API (e.g., the oil, extract, mixture, etc.) may include an indicator that can be scanned by the personal electronic device and utilized to determine one or more properties of the API. For example, upon scanning the cartridge or container storing the API, the user may be able to access information regarding the API type (e.g., cannabis oil, tobacco oil, inhalable steroid, etc.), target dosage for a particular ailment, and the like. Furthermore, after administration of the API, the user may be asked to provide feedback regarding the efficacy for treating one or more ailments. With the feedback, the target dosage may be continuously modified to provide relief for the one or more ailments. For example, the target dosage may be modified to generate a user dosage specific to one or more individuals utilizing the vaporizing device. Additionally, in certain embodiments, the target dosage may be updated universally for all users that utilize the specific API.

In certain embodiments, the oil mixture includes a cannabis oil having a quantity of active tetrahydrocannabinol (THC) and/or Cannabidiol (CBD) or any other active ingredient. The oil mixture may also include an excipient, such as polyethylene glycol (PEG), added in a quantity proportional to the quantity of active THC and/or CBD in the oil such that cartridge containing a cannabis oil having a higher concentration of THC and/or CBD contains a greater amount of PEG than a cartridge containing a cannabis oil having a lower concentration of THC and/or CBD. It should be appreciated that the active THC and/or CBD can be formulated in a variety of configurations to provide relief to one or more ailments suffered by a user. For example, the oil mixture may have a 1:1 ratio of THC to CBD, a 1:2 ratio, a 1:5 ratio, a 1:50 ratio, a 2:1 ratio, a 5:1 ratio, a 50:1 ratio, or any other reasonable ratio of THC to CBD. As such, the oil mixture can be formulated to enhance the medicinal properties based on the response of one or more ailments.

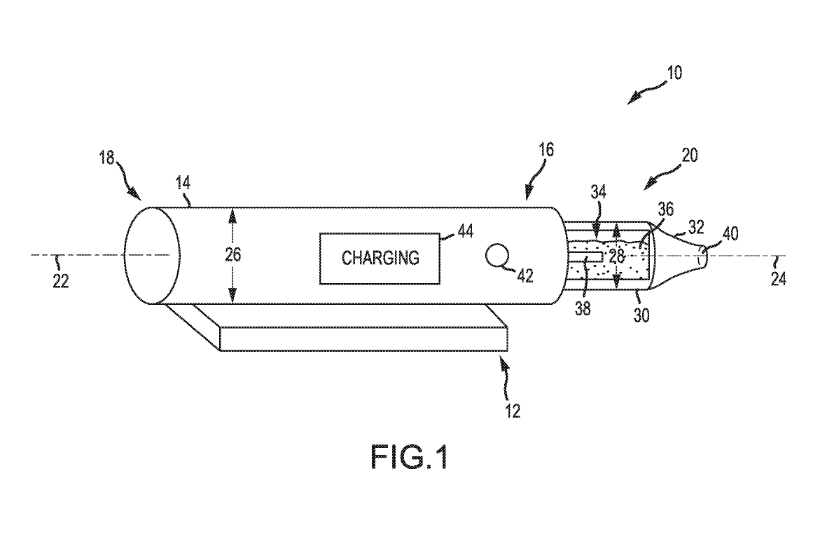

FIG. 1 is a front perspective view of an embodiment of a vaporizing device 10 positioned in electrical communication with a charging element 12. In the illustrated embodiment, the charging element inductively couples to a battery positioned within a body portion 14 of the vaporizing device 10 to provide electrical power to the battery. The body portion 14 is substantially cylindrical and has a proximal end 16 and a distal end 18, in the illustrated embodiment. However, it should be appreciated that in other embodiments the body portion 14 may be rectangular, triangular, or any other suitable shape. In the illustrated embodiment, a cartridge portion 20 is coupled to the proximal end 16 (e.g., via threads, via fasteners, etc.) and substantially aligned with the body portion 14. That is, a body portion axis 22 and a cartridge portion axis 24 are substantially coaxial. In other words, the body portion 14 and the cartridge portion 20 are linearly and axially adjacent in the illustrated embodiment. Yet, it should be appreciated that in other embodiments the body portion axis 22 and the cartridge portion axis 24 may not be aligned.

In the illustrated embodiment, a body portion diameter 26 is larger than a cartridge portion diameter 28. However, in other embodiments, the body portion diameter 26 may be smaller than the cartridge portion diameter 28, or substantially the same size as the cartridge portion diameter 28. As shown, the cartridge portion 20 includes a cartridge body 30 and a mouthpiece 32 arranged adjacent and fluidly coupled to the cartridge body 30. The cartridge body 30 is generally cylindrical and includes a chamber 34 to store an active pharmaceutical ingredient (API), such as an oil or extract 36 (e.g., an oil mixture, cannabis oil, tobacco oil, other inhalable medicines etc.) in preparation for vaporization and inhalation. However, it should be noted that, in certain embodiments, the API may not be the oil 36. For example, the API may include a powdered inhalable substance (e.g., an anti-inflammatory or steroid), or a mist (e.g., an inhalable vaccine, an anti-inflammatory or steroid). In the illustrated embodiment, a wick 38 extends into the chamber 34 to transport the oil or extract 36 from the chamber 34 and toward a heating component stored in the body portion 14 and arranged at the proximal end 16. However, in other embodiments, the chamber 34 may be formulated to not utilize the wick 38. For example, the wick 38 may not be used when the API is a powdered inhalable substance. The heating component transfers energy to the oil 36 to convert the oil 36 into a vapor that can be inhaled by a user through the mouthpiece 32 when the vaporizing device 10 is in a use condition. That is, the heating component provides energy, in the form of heat, to the oil 36 to release the activate ingredients (e.g., THC, nicotine, etc.) without combusting the oil 36. In this manner, the user can activate the vaporizing device 10 such that the vaporizing device 10 is in the use condition and administer a dosage of the activate ingredient from the oil 36. As described above, the mouthpiece 32 is fluidly coupled to the cartridge body 30 such that vapor generated by the heating component can travel through an opening 40 in the mouthpiece 32 and to the user for inhalation. Furthermore, in certain embodiments, the mouthpiece 32 enables the inhalable powder or mist to be directed from the chamber 34 to the user.

In the illustrated embodiment, the body portion 14 includes a switch 42 arranged at the proximal end 16 of the body portion 14. However, the position of the switch 42 may be changed along any portion of the body portion 14 to enable users to activate the vaporizing device 10. In certain embodiments, the switch 42 can function as an ON/OFF switch to provide electrical energy to the heating component. That is, the switch 42 may be coupled to a battery that provides electrical power to the heating component. Upon activation, the switch 42 may enable the battery to provide the electrical energy to the heating component to facilitate conversion of the oil 36 into the vapor for inhalation by the user. Moreover, in certain embodiments, the switch 42 may be utilized to deactivate the heating component. In this manner, the user may have improved control over the vaporizing device. Additionally, as will be described below, the switch 42 may also be utilized to trigger a timing device (e.g., a stop watch) to record an inhalation duration of a user while using the vaporizing device 10. For example, the user may activate (e.g., press) the switch 42 at the beginning of the inhalation and then press the switch 42 at the end of the inhalation. Moreover, in certain embodiments, the user may press and hold the switch 42 for the duration of the inhalation. In this manner, the duration of the inhalation may be recorded for later evaluation and processing. Furthermore, in certain embodiments, the switch 42 may be utilized to provide feedback to the user. For example, the switch 42 may be illuminated to indicate the vaporizing device 10 is on or that battery charging is complete. As another example, the switch may illuminate to inform the user to begin inhaling and provide another illumination or turn off the illumination when the user it stop inhaling. This may be based on a lapse of time or based on the amount of API inhaled or based on any other indicia. Additionally, the switch 42 may be utilized to pair the vaporizing device 10 to a personal electronic device, such as via a BLUETOOTH transceiver or other wireless communication system. In this manner, the user may interact with the switch 42 to transmit and receive information related to using the vaporizing device 10.

Still referring to FIG. 1, the illustrated body portion 14 includes a display 44 arranged on the surface. For example, the display 44 may be an organic light emitting diode (OLED), light emitting diode (LED), liquid crystal display (LCD), or any other type of electronic display. The display 44 may be utilized to provide one or more indications to the user during operation of the vaporizing device 10. For example, the display 44 may indicate a countdown to inform the user of an inhalation duration. Additionally, the display 44 may provide an indication that the vaporizing device 10 is on or off, the battery level remaining, the connectivity of the vaporizing device 10 to one or more other devices, or the like. The display 44 can provide many different types information and or instructions or other communications, for example, but not limited to, information about the device itself, information about the cartridge, information about the target dosage, information about the inhalation event, information about the user, etc. For example, the display may function like a stop watch or clock to show time lapsing or may be provide instructions to the user such as when to inhale and when to stop inhaling, or it may provide the results of the inhalation event, such as how long the event occurred or how much API was inhaled, etc. Accordingly, the user may quickly look to the display 44 to receive information about the state of the vaporizing device 10 or to receive information about other related aspects.

Devices of the invention can also be configured for use as a “nebulizer” to deliver API in an aerosolized form. A nebulizer is a drug delivery device used to administer medication in the form of a mist inhaled into the lungs. Nebulizers can aerosolize medicine through various mechanisms such the use of oxygen, compressed air or ultrasonic power to break up medical solutions and suspensions into small aerosol droplets that can be directly inhaled from the mouthpiece of the device. A nebulizer can provide action by mechanical means (such as a spring); or by electrical means (such as vibrating mesh technology, jet nebulizers (atomizers), and ultrasonic wave nebulizers). In such an embodiment, devices of the invention are configured so that the heating component 60 is replaced or is used in conjunction with an appropriate component to provide the mechanical or electrical means for aerosolizing the API into a mist.

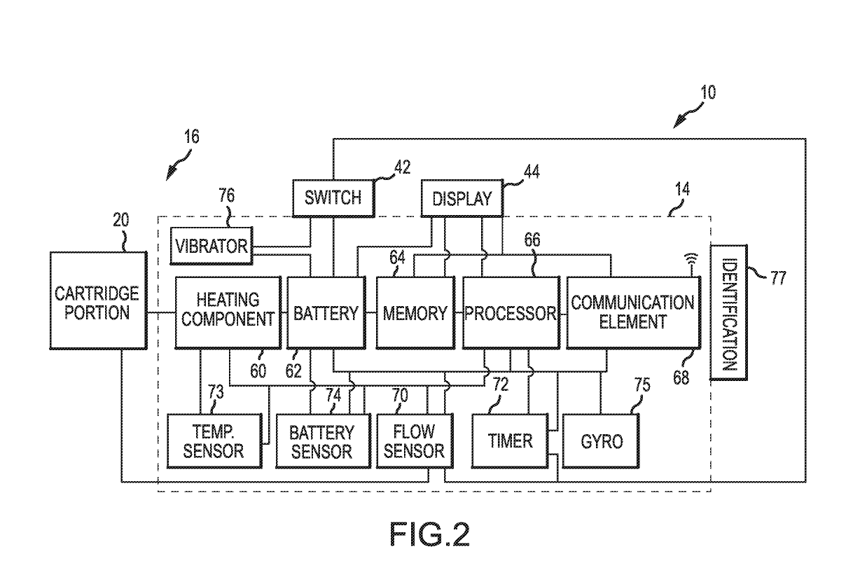

FIG. 2 is a schematic diagram of an embodiment of the vaporizing device 10. In the illustrated embodiment, a heating component 60 is arranged at the proximal end 16 of the body portion 14, thereby abutting the cartridge portion 20. As described above, the heating component 60 transmits energy in the form of heat to the cartridge portion 20 to convert the oil 36 into vapor for inhalation by the user. In certain embodiments, the heating component 60 transmits the energy via conductive heat transfer to the oil 36. However, in other embodiments, the heating component 60 may utilize convective heat transfer by utilizing hot air to raise the temperature within the chamber 34, and thereby heat the oil 36 to enable conversion to vapor. In the illustrated embodiment, the heating component 60 is electrically coupled to and adjacent a battery 62 such that the battery 62 can supply electrical power to the heating component 60. In certain embodiments, the battery 62 is a lithium-ion battery that can be inductively coupled to the charging element 12. For example, the battery 62 may be coupled to a coil that interacts with a coil of the charging element 12 to wirelessly transmit electrical power from the charting element to the battery 62. Moreover, it should be noted that, in certain embodiments, the heating component 60 may be utilized to activate and/or drive the API out of the chamber 34 and toward the user. For example, the cartridge portion 20 containing the API associated with a powdered or mist inhalable product may include a plunger and/or thermal switch that is activated by the heating component 60. The thermal switch may be heated by the heating component 60 until a certain temperature is reached, thereby activating the thermal switch and driving the API out of the chamber 34 and toward the mouthpiece 32. Furthermore, in certain embodiments, the heating component 60 may not be utilized to activate and release the API in the chamber 34. For example, the chamber 34 may include a spring-loaded activator that directs the vaporized (e.g., gas, mist, powdered, etc.) API toward the mouthpiece 34 via movement of the body portion 14 toward the cartridge portion 20.

In the illustrated embodiment, a battery 62 provides electrical energy to the components in the body portion 14. For example, the battery 62 is electrically coupled to a memory 64, a processor 66, and a communication element 68. In the illustrated embodiment, the memory 64 is a non-transitory computer-readable media, which may include non-volatile memory, such as read-only memory (ROM), EEPROM, and/or flash memory which may be used in conjunction with volatile memory, such as Dynamic Random Access Memory (DRAM) and/or Static Random Access Memory (SRAM). Further, the memory 64 can include written instructions (e.g., programs) to be executed by the processor 66. In certain embodiments, the processor 66 includes one or more micro-processors that perform the machine-readable instructions printed on the memory 64. For example, the memory 64 may include instructions to communicate to the heating component 60 regarding the temperature or duration of heating event, for example, at which the heating component 60 will heat the oil 36. Therefore, upon activation, the processor 66 may send a signal to the heating component 60 to operate at the programmed temperature or time. In the illustrated embodiment, the communication element 68 is electrically coupled to the battery 62 and communicatively coupled to at least the processor 66 and the memory 64. In certain embodiments, the communication element 68 includes a BLUETOOTH transceiver, a near field communication (NFC) transceiver, a wireless internet transceiver, or a combination thereof. As used herein, transceiver refers to a device capable of sending and receiving communication signals. As will be described in detail below, the communication element 68 is positioned to communicate (e.g., transmit and/or receive) with one or more personal electronic devices to relay information such as dosing information indicative of the oil 36 being utilized, the therapeutic dose, or the like.

In certain embodiments, the user engages the switch 42 (e.g., presses or slides the switch 42) to activate the battery 62 to bring the vaporizing device 10 into the use condition. As used herein, the use condition refers to state in which the vaporizing device 10 is converting the oil 36 into vapor for inhalation by the user. Upon activation, the battery 62 turns on the heating component 60 to generate heat, via conductive or convective heat transfer, toward the oil 36 to convert the oil 36 into vapor for inhalation by the user. Once the user is complete and has received the dosage, the user may engage the switch 42 a second time, thereby deactivating the battery 62, and as a result, the heating component 60. Additionally, in certain embodiments, the switch 42 may be utilized to time an inhalation duration. For example, the user may press and hold the switch 42 throughout the inhalation, and release the switch 42 after the inhalation is completed. In this manner, the inhalation duration may be monitored and recorded for future evaluation. In this manner, the user may control the operation of the vaporizing device 10.

Furthermore, in certain embodiments, the switch 42 may provide an indication to the user regarding one or more properties of the vaporizing device 10. For example, in certain embodiments, the switch 42 may be illuminated when the vaporizing device 10 is in the use condition to provide an indication to the user that the heating component 60 is supplying heat to the chamber 34. Moreover, other indicia may be utilized to instruct the user on the duration of inhalation (e.g., provide a countdown) to obtain the dosage (e.g., actual dosage, target dosage, user dosage, etc.). For example, upon detecting the user inhaling at the mouthpiece 32 (e.g., via a flow sensor 70), the switch 42 (or another indicator, such as the display 44) may be illuminated as green (e.g., a start indicia), switch to yellow after a period of time, and then switch to red as the period for inhalation comes to an end (e.g., an end indicia). In this manner, the user will know how long to inhale, thereby increasing the likelihood that the user receives the full dosage. Moreover, while the above described indicia is an illumination of the switch 42, in other embodiments the display 44 may be illuminated, or some other indicator, such as the tip of the body portion 14 and/or a personal electronic device screen that is communicatively coupled to the vaporizing device 10 may be illuminated. Furthermore, other indicators may be utilized, such as sounds, vibrations, or any other method to provide an indication to the user.

As described above, in certain embodiments the vaporizing device 10 includes the flow sensor 70 to detect when the user begins inhalation at the mouthpiece 32. For example, the flow sensor 70 may include orifice plates with associated pressure sensors, thermal mass flow meters with associated temperature sensors, a turbine flow meter, a floating orifice disk, or the like. For example, the cartridge portion 20 and/or the body portion 14 may include a floating orifice disk in a flow path. The floating orifice disk may be acted upon by the inhalation of the user, thereby applying a force to the floating orifice disk. In certain embodiments, a magnetic sensor may be coupled to the floating orifice disk and a wall of the flow path. As the force acts on, and moves, the floating orifice disk, the magnetic sensor may be activated and relay a signal to the processor 66 indicative of a flow and/or inhalation event at the mouthpiece 32. In this manner, the vaporizing device 10 can detect when the user begins the inhalation event. As illustrated, the flow sensor 70 is communicatively coupled to the processor 66, the communication element 68, the switch 42, and a timer 72. Accordingly, information acquired via the flow sensor 70 can be transmitted and utilized by the processor 66.

Moreover, as illustrated in FIG. 2, the vaporizing device 10 includes the timer 72. The timer 72 can record the inhalation duration, time of day, or the like. By utilizing the timer 72 with other components of the vaporizing device 10 (e.g., the processor 66) the total dosage administered by the user on a daily basis may be determined. For example, each day, the timer 72 may record the inhalation duration and transmit the information to the communicatively coupled processor 66. Thereafter, the processor 66 may determine the active ingredients (e.g., THC, tobacco, anti-inflammatory medication, etc.) administered to the user based on the inhalation duration. In this manner, the processor 66 may sum the total amount of active ingredients each day, thereby calculating the daily dosage administered by the user. This daily dosage may be transmitted, via the communication element 68, to one or more servers or medical professionals to enable tailoring and modification to the user's prescribed medicinal treatment. As such, each user may have a customized treatment plan based on their usage and subsequent response to the API administered by the vaporizing device 10. Moreover, by monitoring the daily dosage, the total amount of API remaining in the cartridge portion 20 may be monitored, thereby enabling notifications to the user to purchase additional cartridge portions 20 when running low. In certain embodiments, the timer 72 may record inhalation duration in a period of seconds. However, in other embodiments, the period may be in fractions of a second, such as a half of a second, a quarter of a second, a tenth of a second, or the like. As a result, greater precision may be utilized to administer and monitor the API via the vaporizing device 10.

In the illustrated embodiment, a temperature sensor 73 is communicatively coupled to the heating component 60 and the processor 66. As shown, the temperature sensor 73 is positioned to monitor the temperature of the heating component 60. In certain embodiments, different formulations of API may vaporize at different temperatures. For example, certain formulations may vaporize at lower temperatures than others. To that end, the temperature sensor 73 may monitor the heating component 60 to ensure that the desired temperature is utilized to vaporize the oil 36.

The timer and temperature sensor provide precision to the dosage. There may be multiple sensors to provide greater precision in achieving a desired temperature or desired time to heating and there may be multiple sensors to accurately track the inhale event.

FIG. 2 also includes a battery sensor 74 communicatively coupled to the battery 62, the processor 66, and the communication element 68. As shown, the battery sensor 74 monitors the storage capacity of the battery 62. For example, the battery sensor 74 can determine the remaining energy in the battery 62 and communicate the information to the processor 66 for display on the display 44. In this manner, the user can be informed of the remaining life in the battery 62 and whether or not to place the vaporizing device 10 on a charging element 12.

Furthermore, in the illustrated embodiment, the vaporizing device 10 also includes a gyroscope 75. As illustrated, the gyroscope 75 is positioned within the body portion 14 and is communicatively coupled to the processor 66 and the communication element 68. The gyroscope 65 is used to measure orientation of the vaporizing device 10. For example, the gyroscope 65 may be utilized to determine whether the vaporizing device 10 is in use. In certain embodiments, the gyroscope 65 may determine that the vaporizing device 10 is resting on a surface. As such, the processor 66 may instruct the vaporizing device 10 to shut down in order to conserve battery power or prevent the oil 36 from being converted into the vapor when the device is not in use, thereby reducing waste of the oil 36. Additionally, the gyroscope 65 may detect that the vaporizing device 10 is in use (e.g., via the orientation or the movement) and activate one or more features of the vaporizing device 10 (e.g., the heating component 60, the communication element 68, etc.).

As described above, in certain embodiments the vaporizing device 10, via the switch 42 and/or the display 44, may relay information to the user indicative of one or more operating conditions of the vaporizing device 10. For example, in certain embodiments, the switch 42 may be coupled to a vibrator 76 that provides haptic feedback to the user interacting with the switch 42. In certain embodiments, the vibrator 76 may cause the vaporizing device 10 to vibrate indicative of an event. For example, the vaporizing device 10 may vibrate to indicate a low battery level, a time of day to administer the therapeutic dosage, or the like. As such, additional feedback can be provided to the user utilizing the vaporizing device 10.

Furthermore, the illustrated embodiment includes an identification 77. For example, the identification 77 can be a unique, traceable code and/or number that corresponds to the user authorized to use the vaporizing device 10. In certain embodiments, the identification 77 may be arranged on the body portion 14 of the vaporizing device 10. However, in other embodiments the identification 77 may be printed on the interior of the body portion 14, or may be incorporated into one of the components. Accordingly, access and/or use of the vaporizing device 10 can be restricted, for example, in embodiments where the API is THC.

The components making up the vaporizing device 10 enable use for a variety of treatments and ailments. For example, inhalation of cannabis for medicinal purposes may be monitored and tracked utilizing the vaporizing device 10. By closely monitoring dosages, dosage intervals, and efficacy, improved treatment plans may be developed for users. Moreover, in certain embodiments, other APIs may be utilized to treat one or more ailments. For example, in certain embodiments, the API may be nicotine and the vaporizing device 10 may be utilized as a cessation device to help the user quit smoking cigarettes. The cartridge portion 20 may be formulated to provide a predetermined quantity of nicotine per dosage. That is, each inhalation duration may provide a certain quantity of nicotine to the user. By utilizing the processor 66 and the timer 72, for example, the vaporizing device 10 may determine at which time of the day the user administered the dosage of nicotine. As such, the treatment plan for the user may recommend cessation for a period of time (e.g., one hour). Therefore, the processor 66 may instruct the heating component 60 to remain deactivated until the timer 72 indicates the period of time has passed. In this manner, the user utilizing the vaporizing device 10 may be prevented from administering an additional dosage until the period of time has passed. Moreover, as will be described below, the target dosage and/or the user dosage may be decreased over time as the user stops smoking. As such, the nicotine delivered to the user will be less as time progresses, thereby enabling the user to progressively decrease nicotine inhalation and eventually quit smoking.

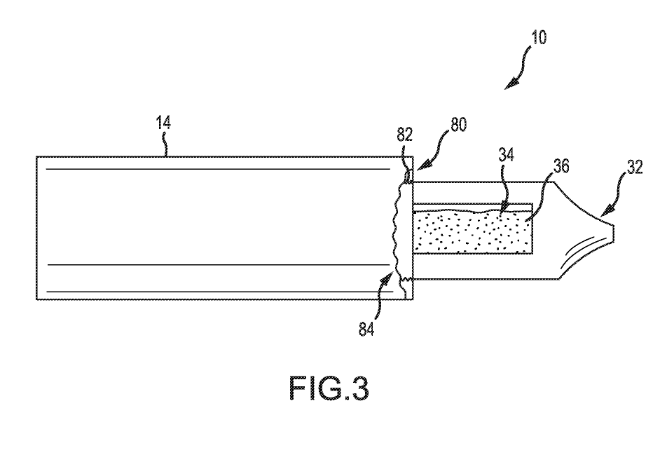

FIG. 3 is a schematic side view of an embodiment of the vaporizing device 10. In the illustrated embodiment, the cartridge portion 20 is coupled to the body portion 14 at a first end 80. For example, in the illustrated embodiment, the first end 80 includes a threaded fitting 82 that couples to a receptor 84 positioned in the body portion 14. In this manner, the first end 80 is positioned proximate to the heating component 60, thereby enabling the transmission of heat from the body portion 14 to the chamber 34 to vaporize the oil 36. Furthermore, in certain embodiments, the cartridge portion 20 and/or the body portion 14 may include one or more features to block authorized/non-compatible devices from being utilized with the body portion 14. For example, the body portion 14 may include a ridge that aligns with a corresponding slot of the cartridge portion 20 to enable the cartridge portion 20 to be used with the body portion 14.

In the illustrated embodiment, the chamber 34 is filled with the oil 36. As used here, oil refers to oils or oil mixtures. For example, the oil mixture may include extracts or oils (e.g., cannabis oils, tobacco oils, etc.) or other APIs (e.g., steroids, anti-inflammatory medication, vaccines, etc.) and an excipient, such as polyethylene glycol (PEG). For example, the excipient may be PEG 400, PEG 3350, or any other reasonable formulation. In the illustrated embodiment, when administering THC for therapeutic purposes (e.g., medicinal purposes), a predetermined dose (e.g., a target dosage) may be desired to treat one or more ailments utilizing a quantity of active THC. Accordingly, the quantity of PEG is proportional to the quantity of active THC such that the oil 36 in the cartridge has higher quantities of PEG for cannabis oils having higher quantities of active THC and lower quantities of PEG for cannabis oils having lower quantities of active THC. Similar techniques may be utilized for other oil, extracts, or medications utilized with the disclosed system. In this manner, each cartridge portion 20 may be manufactured such that the chamber 34 contains the oil 36 having a predetermined amount of active ingredient, such as example, approximately 200 mg of active THC. However, in other embodiments, the cartridge portions 20 may be manufactured to have different levels of active THC. For example, the oil 36 in the chamber 34 may include approximately 50 mg of active THC, approximately 100 mg of active THC, approximately 150 mg of active THC, approximately 250 mg of active THC, or any other suitable amount of active THC. Moreover, as used herein, approximately means no more than plus or minus five percent. Therefore, the chamber 34 containing the oil 36 having approximately 200 mg of active THC may include a range of active THC between 190 mg and 210 mg. As will be described below, by formulating the oil 36 to have a specific quantity of active THC, the dosage may be predetermined based on a duration of inhalation by the user. For example, in certain embodiments, the oil 36 may be formulated such that an eight second inhalation transmits approximately 1.5 mg of active THC to the user. It should be noted that different formulations may transmit different quantities of API to the users. For example, in embodiments utilizing THC as the active ingredient, the oil 36 in the chamber 34 may be formulated to transmit approximately 1 mg to 2 mg of active THC to the user per eight second inhalation or any desired amount of active ingredient per a set time period for the duration of inhalation.

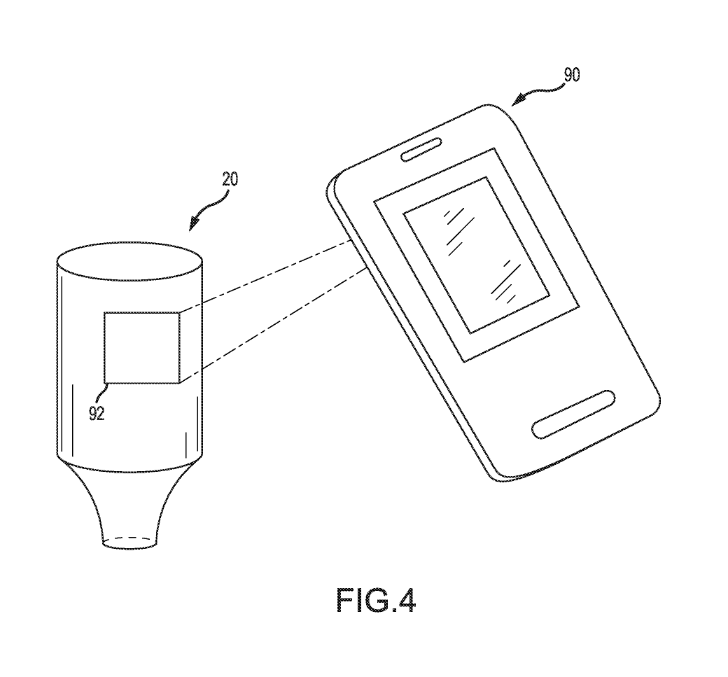

FIG. 4 is a schematic perspective view of an embodiment of a personal electronic device 90 (e.g., a smart phone) scanning the cartridge portion 20. In the illustrated embodiment, the personal electronic device 90 is a cellular phone having a camera that scans a label 92 (e.g., an indicator) arranged on the cartridge portion 20. While the illustrated embodiment includes the label 92 on the cartridge portion 20, in other embodiments the label 92 may be on a container or package that holds the cartridge portion 20, or any other suitable location. The label 92 may include a matrix bar code (e.g., QR Code), an RFID tag, a linear bar code (e.g., UPC), or any other suitable interface to enable wireless transmission of information between the label 92 and the personal electronic device 92. The information transmitted to the personal electronic device 90 may include the strain of plant (e.g., cannabis, tobacco, etc.) utilized to make the oil 36, the quantity of active ingredient (e.g., THC, nicotine, steroid, anti-inflammatory, vaccine, etc.) in the oil 36, the symptoms the oil 36 treats, the recommended therapeutic dose, the user dosage, or the like. For example, in certain embodiments, the label 92 may transmit the name of the strain utilized to form the oil 36 to the personal electronic device 90. There, the personal electronic device 90 may communicate with a server to determine recommended dosing information for a given user. For example, the name of the strain may correspond to a number of medical conditions and symptoms that may be treated for a given user. In this manner, scanning the label 92 before using the cartridge portion 20 can relay dosing information to the user for better management and administration.

Moreover, utilizing the personal electronic device 90 with the vaporizing device 10 also simplifies the reporting and/or logging regulations that are often placed on users of medicinal cannabis. By integrating the logging with the application utilized to operate the vaporizing device 10, the user can easily track their usage, monitor their symptoms, and receive notifications for refills.

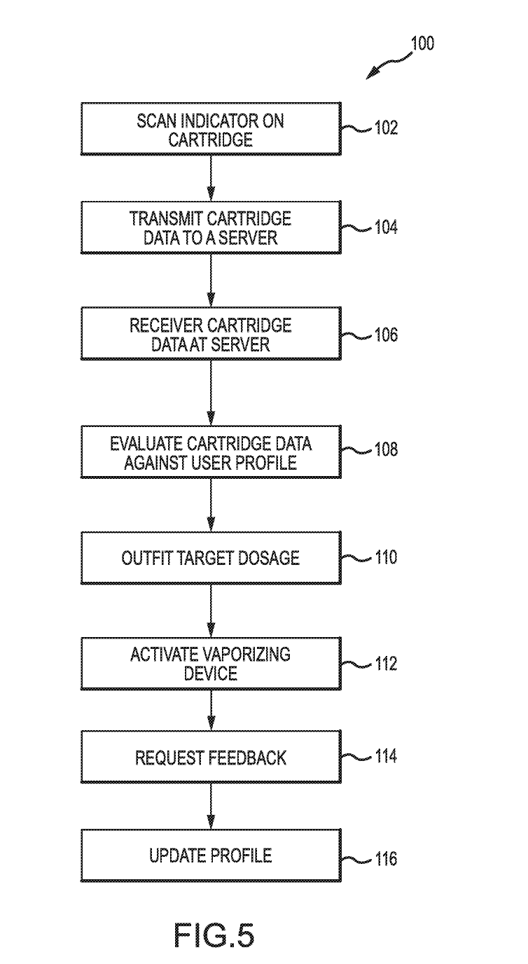

FIG. 5 is a flow chart of an embodiment of a method 100 for administering a dosage with the vaporizing device 10. As used herein, dosage refers to a measure of time which corresponds to a time in which the heating component 60 is activated. This time of activation is directly correlated to the amount of the oil 36 converted into vapor for inhalation. Moreover, in certain embodiments, dosage refers to administration of the API via the vaporizing device. Specifically, when utilizing one or more cartridge portions 20 having the oil 36, the user may be administering a target dosage, a user dosage, an actual dosage, or the like to treat one or more ailments. As used herein, target dosage refers to an initial and/or stored therapeutic dosage prescribed to treat a certain ailment with a certain prescribed amount of active ingredient. The target dosage may take into account the strains used and their corresponding amounts of active ingredients. The target dosage may be developed based on predicted efficacy of the APIs, past medical treatments provided to one or more users, or the like. For example, in certain embodiments, the target dosage may refer to a therapeutic dosage of active THC. Yet, it should be appreciated that similar steps may be utilized to transmit therapeutic dosages of nicotine, anti-inflammatory medications, or the like. The target dosage is developed based on the target ailment suffered by the user and the oil 36 utilized by the user. For example, different oils 36 may have different target dosages for the same ailment. As will be described in detail below, the target dosage may be modified over time based on feedback from a plurality of users related to the efficacy of the initial target dosages.

An actual dosage is the dose inhaled by the user. For example, a user may inhale the target dosage, the user dosage, or may inhale for a shorter period of time and receive less than the target dosage and/or user dosage. With devices and method of the invention as described herein, the user will know what his actual dosage was and can provide feedback regarding efficacy and in turn can be used to refine target or user dosages if necessary. Moreover, the user can have the option of administering several dosages (e.g., actual dosages lower than the target dosage) over a period of time to make administering the target dosage and/or user dosage easier. For example, the user may be prescribed a target dosage and/or user dosage correlating to an 8-second inhalation. However, the user may have difficulty inhaling for the entire 8-seconds. Because of the monitoring provided by the vaporizing device 10, the user can choose to administer two 4-second inhalations, four 2-second inhalations, or any combination of actual dosages to fully administer the target dosage and/or user dosage. To this end, the user has greater flexibility for administering the target dosage or the user dosage as will be described below.

The label 92 of the cartridge portion 20 is scanned by the personal electronic device 90 (block 102). For example, as illustrated in FIG. 4, the personal electronic device 90 may utilize a camera or some near field communication methods to analyze the label 92 to receive cartridge data indicative of at least one property of the oil 36 within the chamber 34. For example, the cartridge data stored on the label 92 may include information regarding the strain used to formulate the oil 36, the target dosage of the oil 36, the ailments intended to be treated with the oil 36, or any other information relevant to utilizing the oil 36. In this manner, information about the oil 36 may be transmitted to the personal electronic device 90 and may be readily displayed to the user.

In certain embodiments, the cartridge data is transmitted to a server (block 104). For example, the personal electronic device 90 may communicate with the server wirelessly, such as via Wi-Fi or a cellular data network. The server may be positioned away from the personal electronic device 90 at a stored location to receive information from each user utilizing the vaporizing device 10 (block 106). However, in certain embodiments, the server may refer to the personal electronic device 90. For example, the cartridge data may be transmitted to the personal electronic device 90 via a camera or near field communication interface. There, the personal electronic device 90 may access a database stored on a memory that corresponds to the cartridge data to relay information to the user. For example, after the cartridge portion 20 is scanned by the personal electronic device 90, the personal electronic device 90 may relay information, such as the target dosage, to the user via a screen or audible message.

Upon receipt of the cartridge data, the server (e.g., the dedicated server or the personal electronic device 90) may evaluate whether the cartridge data corresponds to one or more dosage profiles (block 108). In certain embodiments, the dosage profiles correlate to one or more registered users that may utilize the vaporizing device 10. For example, the user may receive a prescription from a medical professional to utilize cannabis for medicinal purposes. Upon receipt of the prescription, the user may establish a user profile to interface with the vaporizing device via the personal electronic device 90. Upon establishing the user profile, the user's ailments and recommended treatment may be evaluated against the dosage profiles to determine the target dosage for the user given a particular oil 36. For example, each oil 36 may have an associated dosage profile comprising one or more target dosages for a given ailment. As a result, the user profile may be compared to dosage profile to determine whether the strain has a target dosage associated with an ailment that the user profile contains.

The target dosage is output to the vaporizing device 10 after it is derived from the dosage profiles (block 110). That is, as described above, the dosage profiles can be analyzed against the user profile to determine the target dosage for the given oil 36 and the ailment of the user. The target dosage output to the vaporizing device 10 corresponds to a predetermined dosage for treating the ailment. In certain embodiments, the server may transmit the target dosage to the personal electronic device 90, which then transmits the target dosage to the vaporizing device 10 (e.g., via the communication element 68). However, in other embodiments, the server may be the personal electronic device 90, which can then transmit the target dosage to the vaporizing device 10. That is, the personal electronic device 90 may evaluate the dosage profiles and output the target dosage. Moreover, in other embodiments, the personal electronic device 90 may display the target dosage on the screen for the user to relay to the vaporizing device 10. In other words, the user may receive the target dosage from the personal electronic device 90 and use the vaporizing device 10 in a manner to obtain the target dosage (e.g., by inhaling for a given period of time). Then, the vaporizing device 10 is activated (block 112). As described above, activation of the vaporizing device 10 can include, at least in part, activation of the heating component 60 to transmit heat to the oil 36 to generate the vapor for inhalation by the user. In certain embodiments, the personal electronic device 90 transmits a signal to the vaporizing device 10 to activate the heating component 60. However, in other embodiments, the user may activate the heating component 60 via the switch 42.

As the heating component 60 is activated, heat is transferred to the chamber 34 to convert the oil 36 into the vapor for inhalation by the user. After the user receives the target dosage, the personal electronic device 90 requests feedback from the user (block 114). For example, the personal electronic device 90 may ask the user a series of questions, which can be answered on the personal electronic device 90, regarding the efficacy of the target dosage. In this manner, the effectiveness and also usage of the inhalable product may be tracked by the personal electronic device 90. In certain embodiments, the questions may ask the user to evaluate their level of pain for intervals after the target dosage (e.g., 10 minutes, 30 minutes, 1 hour, etc.). Moreover, the questions may ask the user to evaluate other symptoms caused by the one or more ailments being treated. Thereafter, the dosage profile and/or the user profile may be updated based on the feedback (block 116) to develop a new user dosage. As used herein, the user dosage refers to a unique dosage based on the user's feedback regarding the efficacy of the target dosage. For example, the server and/or personal electronic device may assign the user dosage (either more or less than the target dosage) based on the user's answers for a given ailment, thereby customizing the user's treatment to improve efficacy. For example, if the user reports that the target dosage is not very effective, the dosage profile may be updated to increase the dosage to treat the user's specific ailment. Moreover, in certain embodiments, the user profile may be updated such that future doses of that oil 36 for the particular ailment will be increased or decreased to provide effective treatment for the particular ailment. In other words, the future recommended dosage for a given oil 36 treating a given ailment will be updated to be the user dosage, instead of the target dosage, to provide improved treatment of the given ailment for that user. In this manner, administering a dosage may be continuously updated to tailor dosages to individual users based on individual oils 36.

Furthermore, in certain embodiments, the vapor pen 10 may be arranged to continuously monitor and report usage of the vapor pen 10 to the server 170 and/or the personal electronic device 90. For example, each dosage (e.g., target dosage, actual dosage, user dosage, etc.) dispensed to the user may be reported along with a time stamp, inhalation duration, the actual dosage administered, and the like. In this manner, the server 170 and/or the personal electronic device 90 may compile data regarding the user's experience with the vapor pen 10 on a daily basis, a monthly basis, a quarterly basis, a yearly basis, or any other time frame. Moreover, obtaining dosage details for each use of the vapor pen 10 enables the server 170 and/or the personal electronic device 90 to analyze the data (e.g., via instructions printed on a memory and performed by one or more processors) to provide improved care for the user. For example, the time between dosages may be evaluated to determine how often the user utilizes the vapor pen 10, which may then be evaluated to modify the user dosage to provide improved care. Furthermore, in certain embodiments, the dosage may be correlated to other events experienced by the user, such as chemotherapy or radiation treatments, to determine whether larger or smaller amounts of API may ease one or more symptoms more effectively right after the other events. For example, based on the data collected by the vapor pen 10, the vapor pen 10 may instruct the user (e.g., via the personal electronic device 90 and/or the vapor pen 10) to administer the dosage immediately after a chemotherapy session to alleviate one or more symptoms.

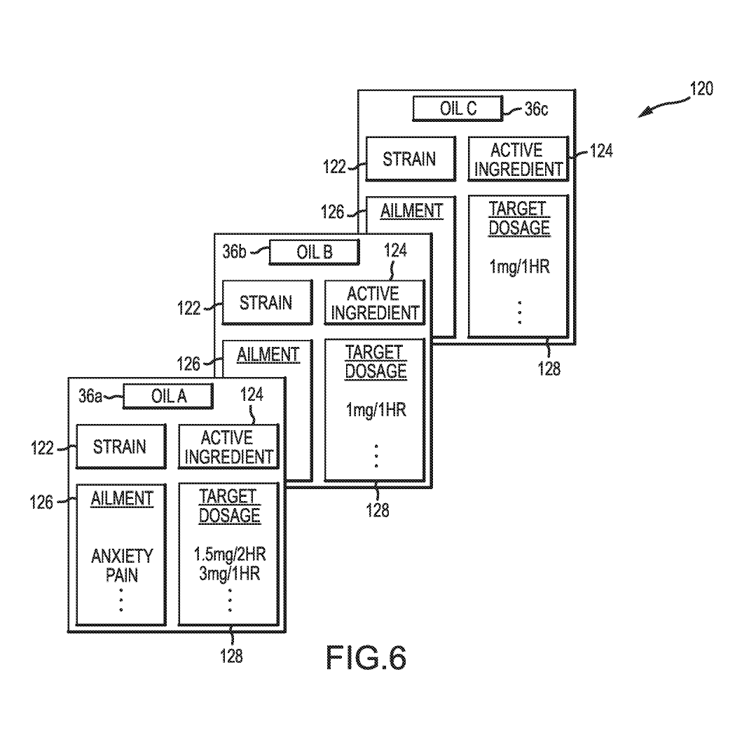

FIG. 6 is a schematic diagram of an embodiment of dosage profiles 120 generated for the oils 36a, 36b, 36c. As shown, each dosage profile 120 may include the strain 122 (e.g., cannabis, tobacco, etc.) utilized to formulate the oil 36 and the quantity of active ingredient 124 in each cartridge portion 20. Moreover, it should be appreciated that, in certain embodiments, the strain 122 can be correlated to the API used to formulate the oil 36, such as an anti-inflammatory or vaccine. For example, as described above, in certain embodiments the oil 36 is formulated to contain approximately 200 mg of active ingredient (e.g., active THC, nicotine, anti-inflammatory, etc.) in each cartridge portion 20. Moreover, in the illustrated embodiment, the dosage profile 120 includes treated ailments 126 and target dosages 128. By way of example only, in the illustrated embodiment for oil 36a, two of the treated ailments include anxiety and pain. Moreover, also by example, the associated target dosages are 1.5 mg every 2 hours and 3 mg every hour, respectively. Accordingly, each oil 36 may have a corresponding dosage profile 120 to effectively categorize and organize ailments 126 and target dosages 128.

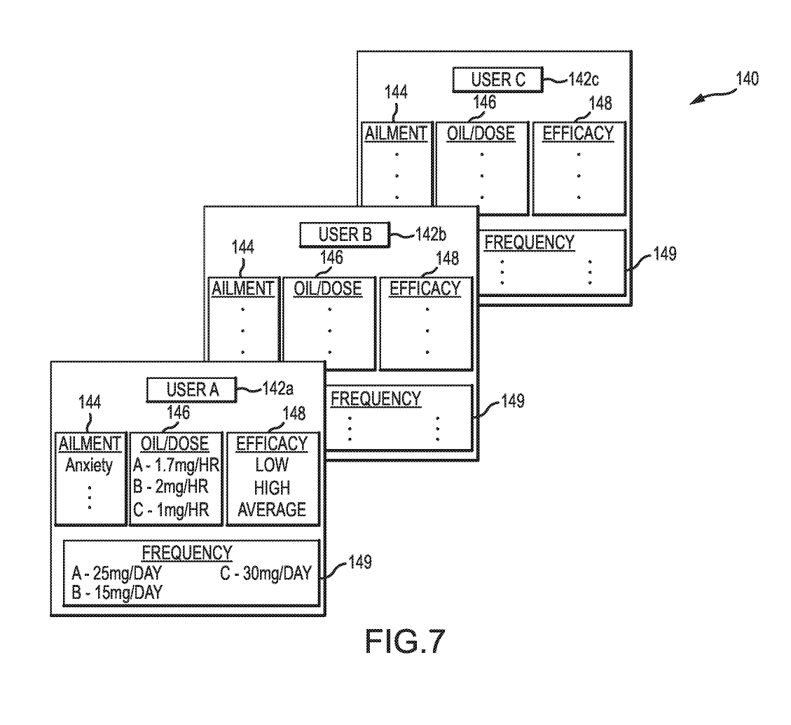

FIG. 7 is a schematic diagram of an embodiment of user profiles 140 generated by an authorized user of the vaporizing device 10. In certain embodiments, the user profile 140 will be generated through a computerized application that is executable on the personal electronic device 90. For example, upon receiving a prescription to utilize medicinal cannabis from a licensed medical professional, a user 142 (e.g., user 142a, user 142b, user 142c) may receive a passcode to enable the download and installation of the application. Thereafter, the user 142 may establish the user profile 140 to list the ailments 144 to be treated by the medicinal cannabis and/or other API. It should be noted that, while the illustrated embodiment includes treatment utilizing medicinal cannabis, in other embodiments the API may be tobacco, an anti-inflammatory, a vaccine, or any other suitable API that can be vaporized and/or inhaled by the user. By way of example only, in the illustrated embodiment, anxiety is listed as an ailment for the user 142a. However, any other number of ailments may be associated with the user profile 140. Moreover, in the illustrated embodiment, each ailment 144 has an associated oil/dose identifier 146. That is, the oil/dose identifier 146 lists the oils 36 and the user dosages utilized by the user 142 for the given ailment 144. As described above, the user dosage is the unique dosage each user determines provides the proper efficacy to treat their unique ailment. The user dosage is determined via analysis of the efficacy of different oils 36 and adjustments (e.g., increasing or decreasing the dosage) based on the user's feedback. Moreover, the illustrated user profile 140 includes efficacy 148 for the given oils 36 and dosages identified in the oil/dose identifier 146. In this manner, the user 142 may quickly and efficiently identify the oils 36 and user dosages that have been utilized to treat the given ailment 144. Moreover, the user 142 can identify the efficacy of the oils 36 and user dosages for further diagnosis and refinement by their medical practitioner.

Furthermore, in the illustrated embodiment, a frequency 149 is also included within the user profile 140. The frequency 149 is correlated to the total amount of API (e.g., cannabis, tobacco, anti-inflammatory, etc.) utilized by the user 142 over a period of time. For example, the frequency 149 may measure dosages per day, per week, per month, or any other suitable time frame. In this manner, the user's treatment plan can be continuously monitored and updated by their medical professional. Additionally, the frequency 149 may track each administered dosage of the vapor pen 10 for the user. For example, in certain embodiments, use of the vaporizing device 10 may be unrestricted, thereby allowing the user to administer dosages as often as deemed necessary to treat one or more ailments. The vaporizing device 10 may record and transmit each dosage to the server 170 and/or personal electronic device 90. In certain embodiments, each dosage event includes a date and time stamp. The user profile 140 may save these dosage events to track and evaluate use of the vaporizing device 10 by the user. In this manner, the data collected may be utilized to improve the treatment plan for the user. Furthermore, transmission of the frequency 149 data (e.g., via the communication element 68) may be utilized to remind the user 142 to place an order for more cartridge portions 20. For example, if data indicates that the user 142 purchased the cartridge portion 20 with the oil 36 containing approximately 200 mg of API, and the frequency 149 indicates that the user 142 uses approximately 25 mg per week, it can be extrapolated that the cartridge portion 20 will last approximately eight weeks. Therefore, a notification may be sent to the user 142 (e.g., via the computerized application) to place an order when the supply is running low. Accordingly, the interruption of treatment (e.g., via running out of oil 36) may be reduced, thereby providing improved care to the users 142.

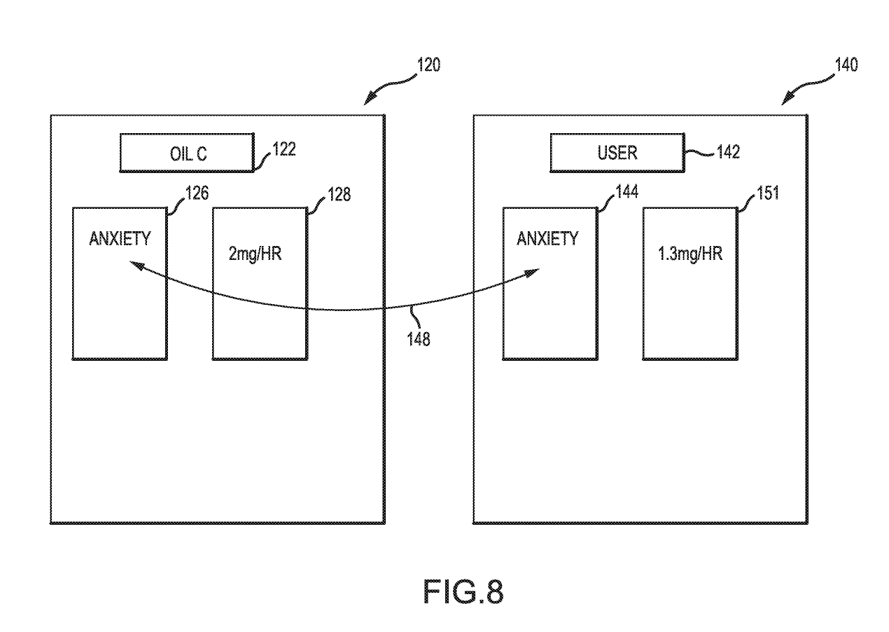

FIG. 8 is a schematic diagram of an embodiment of block 108 in which the method 100 outputs the target dosage 128 derived from one or more dosage profiles 120. As mentioned above, the target dosage 128 is the initial dosage recommended to the user 142 for treatment of one or more ailments 144. As will be described below, after the user 142 (or multiple users 142) provide feedback regarding the efficacy of oils 36, a user dosage 151 may be utilized in place of the target dosage 128 to provide treatment to the user 142. In the illustrated embodiment, the dosage profile 120 is established for the oil 36c. Moreover, as illustrated, anxiety is a treatable ailment 126 listed by the oil 36c, with the target dosage 128 of 2 mg per hour. The user profile 140 also lists anxiety as the ailment 144 to be treated. Accordingly, upon evaluation of the dosage profile 120 for the oil 36c, the method 100 may output, to the personal electronic device 90, the target dosage of 2 mg per hour due to the correlation of ailments 126, 144 between the dosage profile 120 and the user profile 140, as illustrated by the arrow 148. In this manner, oils 36 and user profiles 140 may be analyzed to determine treatment options. Furthermore, upon evaluation of feedback from the user, the user profile 140 may be updated to include the user dosage 151. In the illustrated embodiment, the user dosage 151 is lower than the target dosage 128. For example, the target dosage 128 is listed as 2 mg per hour, while the user dosage 151 is listed as 1.3 mg per hour. As described above, the user dosage can be utilized to customize the treatment plan for the user to improve the efficacy of the treatment. Accordingly, the next time the user administers the dosage of oil 36c, the user dosage 151 will be relayed to the user (e.g., via the personal electronic device 90 and/or the vapor pen 10) instead of the target dosage 128. Thereafter, additional feedback provided regarding efficacy will be based on the user dosage 151 because future dosages will be based on the user dosage 151 instead of the target dosage 128.

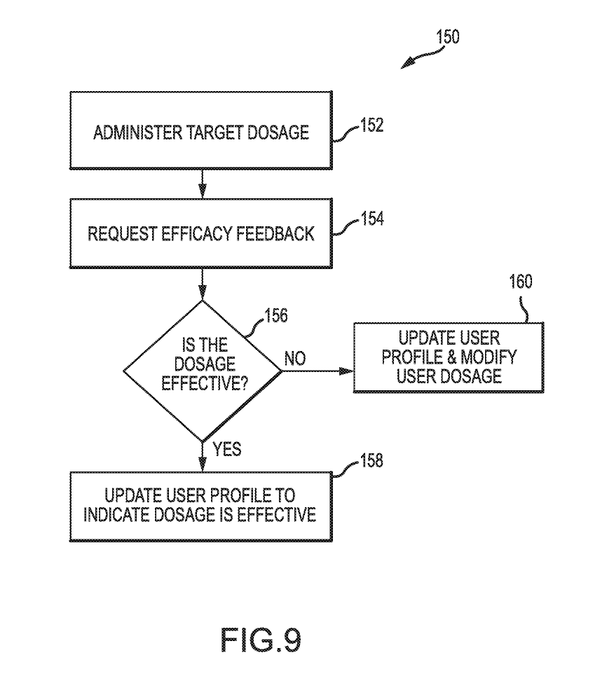

FIG. 9 is a flow chart of an embodiment of a method 150 for administering dosages and creating the user dosage 151. In certain embodiments, the method 150 also includes updating the dosage profile 120 based on feedback from the user 142. As described above, the vaporizing device 10 and the accompanying computerized application associated with the personal electronic device 90 requests feedback regarding the efficacy of the target dosage 128 and/or the user dosage 151 after the user 142 administers the dosage (block 152). For example, the user 142 may activate the vaporizing device 10 via the switch 42 to convert the oil 36 into an inhalable vapor. Moreover, in certain embodiments, the vaporizing device 10 may be activated via a signal from the personal electronic device 90. As described above, the target dosage 128 is the initial recommended dosage to treat one or more ailments 126, 144 utilizing a particular oil 36. After the user 142 administers the target dosage 128, the personal electronic device 90 requests feedback regarding the efficacy of the target dosage 128 (block 154). For example, the screen of the personal electronic device 90 may present one or more questions to the user 142 to determine the efficacy of the target dosage 128. For example, the questions may ask the user 142 to rank their pain on a scale from 1-10, to quantify the anxiety they feel after administering the target dosage, whether or not they continue to experience nausea, or any other question that may be useful in determining the efficacy of the target dosage 128. Furthermore, the questions may be asked over a period of time (e.g., 15 minutes, 30 minutes, 1 hour, etc.) to determine the continual efficacy of the target dosage 128. Moreover, in certain embodiments, the feedback may be passive feedback obtained by monitoring one or more wearable devices coupled to the user 142. For example, the wearable devices may include heart rate monitors, pedometers, or the like that are communicatively coupled to the personal electronic device 90. By monitoring the wearable devices and/or the active feedback input manually by the user 142, the efficacy of the target dosage 128 may be evaluated. Next, the method 150 may evaluate whether the target dosage 128 is effective (operator 156). For example, the user 142 may input their perceived effectiveness on a scale of 1 to 10 to determine the efficacy of the target dosage 128. Moreover, the user may describe the efficacy of the dosage (e.g., low, high, average, etc.). Furthermore, the information obtained from the wearable devices may be evaluated to determine the effect on the user 142. For example, if the ailment were anxiety, the personal electronic device 90 may monitor the wearable device to determine whether or not the user's pulse rate decreased. Based on the feedback from the user 142, a user dosage is created to personalize and/or customize the dosage administered to the user 142 for a given ailment 144 and a given oil 36. If the target dosage 128 is deemed effective, then the user dosage 151 is substantially the same as the target dosage 128. As a result, the user profile 140 is updated to indicate the oil 36 utilized to treat the ailment 144, the dosage (e.g., the user dosage 151), and the efficacy (block 158). However, if the target dosage 128 is deemed ineffective, then the user profile 140 and the user dosage 151 are updated (block 160). For example, the user profile may be updated to indicate that the target dosage 128 for a given oil 36 is not effective at treating the given ailment 144, and therefore the user dosage 151 is created to provide improved treatment. Moreover, the user dosage is modified based on the answers provided by the user 142. For example, if the user 142 indicates that the target dosage 128 has low efficacy, the user dosage 151 may be updated to increase the amount of active ingredient administered to the user 142. Additionally, if the user 142 reports the target dosage 128 is overwhelming, or too much for their ailment 144, the user dosage 151 may be updated to decrease the amount of active ingredient administered to the user 142. In this manner, the user profile 140 and/or the user dosage 151 may be continuously updated to ensure efficacy of the oils 36. Furthermore, in certain embodiments, future dosages of the given oil 36 will be administered based on the user dosage 151, instead of the target dosage 128. For example, the target dosage 128 may be utilized as a baseline or starting point, but thereafter the user dosage 151 can be utilized to provide improved treatment of the one or more ailments. Furthermore, additional feedback may be requested to continuously monitor the efficacy of the user dosage 151. For example, as described above, the user may administer the user dosage 151, after the user dosage 151 is established. Thereafter, the personal electronic device 90 and/or the server 170 may request feedback regarding the efficacy of the user dosage 151. Based on the feedback, the personal electronic device 90 and/or the server 170 may modify the user dosage 151. For example, the user dosage 151 may be increased, decreased, or remain the same to provide improved efficacy and treatment for the one or more ailments. In this manner, the treatment provided to the user may be continuously monitored and improved.

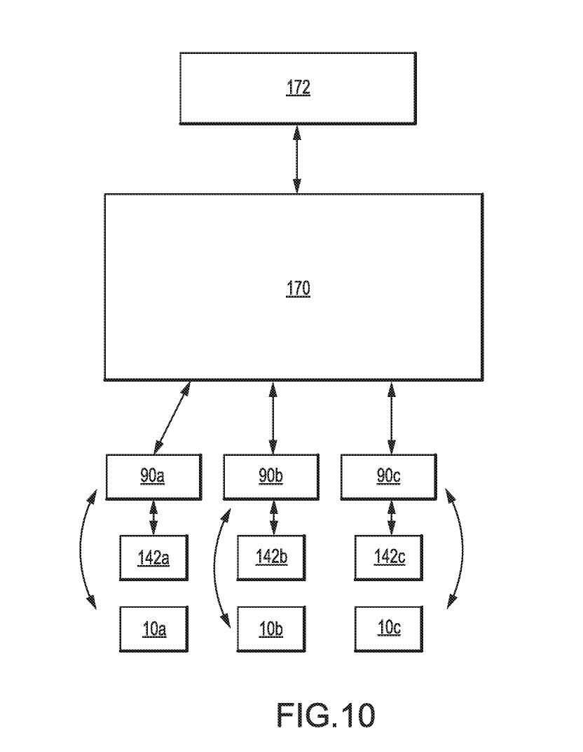

FIG. 10 is a schematic diagram of an embodiment of a server 170 that communicates with one or more personal electronic devices 90. It should be appreciated that the server 170 includes one or more memories and processors capable of utilizing machine-readable code to perform one or more computerized functions. As described above, the server 170 may contain the machine-readable code that includes written instructions to execute a computer application on the personal electronic device 90. Upon receiving authorization to download the computer application (e.g., a prescription to utilize medicinal cannabis, purchasing the vaporizing device 10, etc.), the user 142 sends a signal to the server 170 via the personal electronic device 90 to receive the computer application. Thereafter, the user 142 can interact with the server 170 via the personal electronic device 90 to send and receive information related to administering the target dosage 128 and/or the user dosage 151 via the vaporizing device 10. For example, the user 142 may answer one or more questions regarding the efficacy of the target dosage 128 and/or the user dosage 151. Furthermore, the user 142 may enter information regarding their ailments 144, biometric data (e.g., height, weight, body mass index, etc.), pair one or more wearable fitness devices, or any other information that may be utilized to tailor the target dosage 128 to formulate the user dosage 151 to provide relief for the one or more ailments 144.

Moreover, as illustrated in FIG. 10, the vaporizing device 10 can communicate with the personal electronic device 90 via the communication element 68. For example, in certain embodiments, the personal electronic device 90 may transmit information related to the duration of inhalation by the user 142, the time of day the user 142 administered the target dosage 128, the number of dosages administered by the user 142 over a period of time (e.g., a day, a week, a month), the energy remaining in the battery 62, or any other reasonable information. As a result, in certain embodiments, the personal electronic device 90 may communicate directly with the server 170 to transmit information related to the administration of the target dosage 128 and/or the user dosage 151.

As shown, the server 170 is positioned to receive information from one or more users 142a, 142b, 142c via respective personal electronic devices 90a, 90b, 90c and/or from one or more vaporizing devices 10a, 10b, 10c. Furthermore, the server 170 is also communicatively coupled to a controller 172 that has access to modify one or more properties of the server 170. For example, the controller 172 may be a computer arranged to evaluate the feedback received from the users 142 and/or vaporizing devices 10 to update the dosage profiles 120 and/or provide unique user dosages 151 to the users 142 based on their feedback. However, as described above, in certain embodiments the personal electronic device 90 may include the information (e.g., via downloading database) to evaluate and provide the unique user dosages 151. As a result of having access to the server 170, the controller 172 may evaluate feedback from multiple users each having one or more of the same ailments and each using one or more of the same oils 36 to treat the ailments. By processing the efficacy of certain oils 36 against certain ailments over a number of users over a period of time, the controller 172 may continuously update the dosage profiles 120 to provide relief for the one or more ailments. That is, the target dosage 128 may be adjusted based on feedback from multiple users over a period of time to enhance administration of the target dosage 128 for future users.

As described above, in certain embodiments the vaporizing device 10 transmits information to the personal electronic device 90 and/or to the server 170. For example, when in use, the processor 66 of the vaporizing device 10 may record at least one dosing property, such as duration of inhalation, time of day of dosing, or the like. Thereafter, the communication element 68 may transmit the at least one dosing property to the personal electronic device 90 for processing, evaluation, or record keeping purposes. For example, the dosing property may be the time of day of inhalation. Therefore, the personal electronic device 90 may transmit a signal to the vaporizing device 10 to “lock” or prevent use of the vaporizing device 10 until a certain interval of time has passed. However, in certain embodiments, the vaporizing device 10 may enable unrestricted use. That is, the user may administer as many dosages as the user deems necessary while the vaporizing device 10 monitors and tracks information related to the dosages, such as duration of inhalation, frequency, time of date, and the like. Furthermore, the processor 66 may monitor the duration of inhalation and instruct the heating component 60 to turn off after the recommended duration has been reached. In certain embodiments, the dosage profile 120 and/or user profile 140 may include the recommended inhalation period and that information may be transmitted to the vaporing device 10 via the personal electronic device 90 and/or the server 170. In this manner, the personal electronic device 90 may be utilized to limit and/or regulate the quantity of the active ingredient the user 142 may utilize in a given time period. Furthermore, in certain embodiments, the processor 66 may record the duration of inhalation and transmit a signal to the personal electronic device 90 indicative of the duration. If the duration of inhalation is less than the recommended duration, for example, the personal electronic device 90 may notify the user 142 that the duration of inhalation was not long enough and instruct the user to perform a second inhalation to obtain the target dosage 128 and/or the user dosage 151. In this manner, administration of the target dosage 128 and/or the user dosage 151 can be regulated. For example, because feedback regarding the efficacy of the target dosage 128 and/or the user dosage 151 is requested, monitoring whether or not the user administered the full recommended dosage may be utilized to evaluate the efficacy. That is, if the user reports low efficacy, but only administered a fraction of the target dosage 128 and/or the user dosage 151, the server 170 and/or the personal electronic device 90 may place a lower priority or weight to the feedback because the recommended dosage was not properly administered.

Furthermore, as described above, the server 170 may be utilized to evaluate the feedback and output the unique user dosages 151 to the users 142. For example, upon receiving the feedback from the users 142, the server 170 and/or the controller 172 may analyze the data (e.g., via the one or more processors and memories) to determine whether or not to increase or decrease the dosage. After evaluating the data, the user dosage 151 may be transmitted to the user 142 and the user profile 140 may be updated for certain oils 36. To this end, the user dosage 151 may be continuously updated to provide adequate care for the ailments 144 of the user 142. Furthermore, as described above, in certain embodiments the personal electronic device 90 may evaluate the feedback and update the user profile 140 and/or the user dosage 151 based on the feedback. Accordingly, the user dosage 151 may be continuously monitored and modified based on feedback from the user. For example, because the target dosage 128 is administered as the initial dosage, in certain embodiments future feedback will be related to the efficacy of the user dosage 151, instead of the target dosage 128. While the target dosage 128 may be substantially equal to the user dosage 151, in certain embodiments, continuous feedback may, over time, generate differences between the user dosage 151 and the target dosage 128.

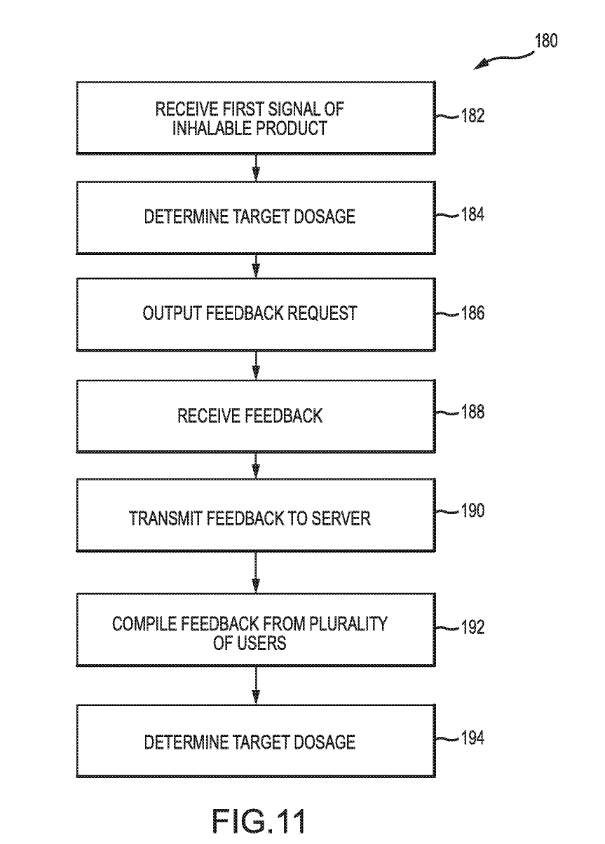

FIG. 11 is a flow chart of an embodiment of a method 180 to determine the dosage (e.g., the target dosage 128) of an inhalable product. In certain embodiments, the inhalable product is the vapor generated by heating the oil 36 of the cartridge portion 20. However, in other embodiments, the inhalable product may be a powder anti-inflammatory, a mist immunization, or any other inhalable API. A first signal indicative of the inhalable product is received (block 182). For example, the personal electronic device 90 can scan the label 92 of the cartridge portion 20 to provide an indication of the oil 36 formulation. Next, the target dosage 128 is determined (block 184). For example, as illustrated in FIG. 8, the dosage profile 120 corresponding to the inhalable product may be evaluated against one or more ailments 144 of the user 142. As the ailment 144 is matched to the ailment 126 treated by the oil 36, the target dosage 128 may be determined.

After administration of the target dosage 128, feedback from the user 142 is requested (block 186). For example, the feedback may be related to the efficacy of the target dosage 128 for the one or more ailments 144 being treated. In certain embodiments, feedback is requested at intervals to determine the duration of relief to the user 142. Furthermore, in certain embodiments, the user 142 ranks the efficacy of the target dosage 128 on a scale (e.g., from 1 to 10) or by another method to quantify the efficacy. In this manner, the user 142 can determine how well the target dosage 128 treats the one or more ailments 144. The feedback is received at the personal electronic device 90 (block 188). For example, the user 142 either actively provides the feedback, or in certain embodiments, the user 142 wears one or more wearable fitness products that are communicatively coupled to and monitored by the personal electronic device 90. Thereafter, the feedback is transmitted to the server 170 (block 190). For example, the server 170 may be accessible by the controller 172 to enable one or more administrators to update and/or change the dosage profiles 120. For example, the dosage profiles 120 may update the target dosage 128 based on feedback from one or more users 142 over a period of time. Next, a plurality of feedback from a plurality of users 142 is compiled based on the ailment 144 and oil 36 being utilized to treat the ailment 144 (block 192). For example, feedback related to treatment of anxiety may be analyzed to determine which oils 36 provided relief to the users 142. Thereafter, the dosage profiles 120 may be updated based on the feedback to determine the target dosage 128 to treat each given ailment 144 (block 194). For example, upon evaluation, the feedback received may demonstrate that a larger or smaller target dosage 128 for a given oil 36 provides better treatment for a given ailment 144. In this manner, the target dosage 128 may be constantly updated (e.g., decreased or increased) to provide relief for the given ailments 144. For example, a new user 142 scanning the cartridge portion 20 may receive a modified target dosage based on the analysis of the feedback received from the plurality of users. As such, the users 142 first dosage may be closer to the eventual user dosage 151 created for the user's unique ailment. However, by updating the target dosages 128 continuously, fewer iterations may be utilized between the initial target dosage 128 and eventually determining the user dosage 151 to effectively treat the user's one or more ailments. Moreover, evaluation of the plurality of feedback may enable formation of a database to better recommend oils 36 for various ailments 144. That is, the personal electronic device 90 and/or the service 170 may recommend new APIs to the users 142 to effectively treat their ailments based on the feedback provided by the plurality of users 142.

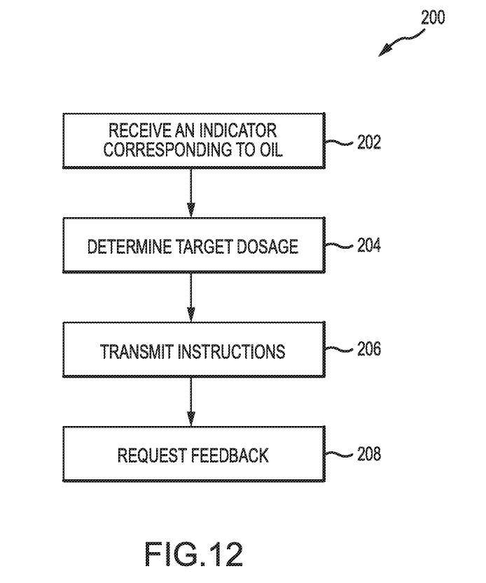

FIG. 12 is a flowchart of an embodiment of a method 200 for administering the target dosage 128 via the vaporizing device 10. In the illustrated embodiment, an indicator corresponding to the oil 36 in the cartridge portion 20 is received (block 202). For example, the personal electronic device 90 may scan the label 92 to determine the strain 122, target dose 128, or the like. Because the label 92 corresponds to the strain 122 and/or API being utilized for the upcoming inhalation, the dosing properties may be targeted specifically to the user 142. Upon receiving the indicator, the target dosage 128 is determined (block 204). In certain embodiments, the indicator is transmitted to the sever 170 for evaluation of one or more dosage profiles 120. However, in other embodiments, the dosage profiles 120 may be loaded onto a memory of the personal electronic device 90. By loading the dosage profiles 120 onto the memory, and periodically updating them as new information is made available, the user 142 can administer the target dosage 128 in areas where an internet connection (e.g., Wi-Fi, cellular, etc.) is unavailable. Once the target dosage 128 is determined, instructions are transmitted for administering the target dosage 128 (block 206). For example, the instructions may appear as a textual display on the personal electronic device 90 providing the inhalation duration that the user 142 should undertake to obtain the target dosage 128. As such, the user 142 can activate the vaporizing device 10 (e.g., via the switch 42) to administer the target dosage 128. Moreover, in certain embodiments, the instructions may be transmitted to the vaporizing device 10 such that the vaporizing device 10 is placed into the use condition to convert the oil 36 into vapor. After the target dosage 128 is administered, feedback is requested from the user 142 (block 208). For example, the personal electronic device 90 may present one or more questions to the user 142 to determine the efficacy of the target dosage 128. In this manner, future dosages administration may be updated and/or evaluated to provide relief for one or more ailments by creating the user dosage, as described above. For example, in certain embodiments, the user dosage may be generated based on the feedback provided by the user 142. As such, future dosages may be based on the user dosage instead of the target dosage.

As described in detail above, embodiments of the present disclosure include the vaporizing device 10 having the body portion 14 coupled to the cartridge portion 20. The cartridge portion 20 includes the chamber 34 to hold the oil 36 for vaporizing and inhalation by the user 142. In certain embodiments, the body portion 14 includes the communication element 68 to send and receive signals from the personal electronic device 90 regarding the operation of the vaporizing device 10. For example, the personal electronic device 90 may send a signal to the vaporizing device 10 to activate the heating component 60 to convert the oil 36 into vapor for inhalation by the user 142. Moreover, in other embodiments, the communication element 68 may transmit dosing information to the personal electronic device 90, such as the duration of inhalation, the time of day of use, and the like. Accordingly, operation of the vaporizing device 10 may be logged and controlled. In certain embodiments, the personal electronic device 90 may request feedback from the user 142 regarding the efficacy of the target dosage 128 administered by the vaporizing device 10. The results of the feedback may be utilized to update the dosage profile 120 for a given strain 122 to improve the treatment of one or more ailments 144. In certain embodiments, a plurality of users may provide feedback for a given ailment 144 and/or strain 122, thereby enabling updates to the dosage profiles 120 to enhance treatment of the one or more ailments 144.

The foregoing disclosure and description of the invention is illustrative and explanatory of the embodiments of the invention. Various changes in the details of the illustrated embodiments can be made within the scope of the appended claims without departing from the true spirit of the invention. The embodiments of the present invention should only be limited by the following claims and their legal equivalents.

1. A vaporizing device, comprising:
a body portion, the body portion comprising:
a heating component positioned at a proximal end of the body portion;
a battery positioned adjacent and electronically coupled to the heating component, the battery providing electrical power to the heating component when the vaporizing device is in a use condition;
a communication element that receives at least one piece of dosing information from at least one portable electronic device;
a processor communicatively coupled to the communication element to evaluate the dosing information utilizing machine-readable instructions stored on at least one memory; and
a cartridge portion positioned linearly adjacent to the body portion to abut the proximal end, the cartridge portion comprising:
a cartridge body coupled to the body portion and having a chamber to hold an active pharmaceutical ingredient (API); and
a mouthpiece arranged adjacent and fluidly coupled to the cartridge body to enable the API to move out of the chamber for inhalation by a user.

2. The vaporizing device of claim 1, comprising a switch arranged on the body portion, the switch being communicatively coupled to the battery to enable the user to activate the heating component or to deactivate the heating component.

3. The vaporizing device of claim 1, comprising a display positioned on a surface of the body portion, the display conveying at least one piece of dosing information to the user.

4. The vaporizing device of claim 1, wherein the communication element comprises a BLUETOOTH transceiver, a near field communication (NFC) transceiver, a wireless internet transceiver, or a combination thereof.

5. The vaporizing device of claim 1, wherein the API comprises an oil and the oil comprises a therapeutic dosage of cannabis or tobacco.

6. The vaporizing device of claim 1, wherein the processor is configured to record at least one dosing property, the dosing property corresponding to one or more parameters of the vaporizing device in the use condition, and to transmit the at least one dosing property to the portable electronic device via the communication element.

7. The vaporizing device of claim 1 comprising an indicator to provide at least one indicia of use to the user when the vaporizing device is in the use condition.

8. The vaporizing device of claim 7, wherein the indicia of use comprises a start indicia, an end indicia, a countdown timer, or a combination thereof.

9. The vaporizing device of claim 1, wherein the API is specifically formulated to deliver a predetermined dosage of an active ingredient at each use of the device.

10. The vaporizing device of claim 1, wherein the chamber comprises a mixture of an oil and an excipient, wherein a volume of excipient in the mixture is proportional to an amount of a pharmaceutically active ingredient in the oil to formulate the mixture to deliver a predetermined dosage of an active ingredient at each use of the device.

11. A vaporizing device, comprising:
a body portion, the body portion comprising:
a heating component positioned within the body portion;
a battery electronically coupled to the heating component within the body portion, the battery providing electrical power to the heating component when the vaporizing device is in a use condition;
a communication element that sends and receives at least one piece of dosing information;
a processor communicatively coupled to the communication element to evaluate the dosing information utilizing machine-readable instructions stored on at least one memory; and
a cartridge portion positioned linearly adjacent to the body portion to abut the proximal end, the cartridge portion containing an activate pharmaceutical ingredient (API) and being coupled to the heating component to receive heat energy from the heating component.

12. The vaporizing device of claim 11, comprising a switch arranged on the body portion, the switch being communicatively coupled to the battery to enable the user to control activation of the heating component, deactivation of the heating component, record a duration of use of the vaporizing device, pair the vaporizing device to a personal electronic device, or a combination thereof.

13. The vaporizing device of claim 11, wherein the API is in the form of an oil that comprises a therapeutic dosage of cannabis or tobacco.

14. The vaporizing device of claim 11, comprising an indicator to provide at least one indicia of use to the user when the vaporizing device is in the use condition.

15. The vaporizing device of claim 11, wherein the communication element comprises a BLUETOOTH transceiver, a near field communication (NFC) transceiver, a wireless internet transceiver, or a combination thereof.

16. A system for administering a dosage of an inhalable product to a user, the system comprising:
a vaporizing device for converting an oil into the inhalable product, the vaporizing device comprising a communication element to send or receive at least one piece of dosage data to or from a portable electronic device, the dosage data corresponding to one or more properties of a target dosage;
at least one processor; and
a memory including machine-readable instructions that, when executed by the at least one processor, cause the system to:
receive an indicator corresponding to the oil utilized by the vaporizing device;
determine the target dosage for the oil utilized by the vaporizing device by evaluating the oil and one or more ailments suffered by the user;
transmit, to the user, instructions for administering the target dosage; and
request feedback from the user regarding the efficacy of the target dosage.

17. The system of claim 16, the machine-readable instructions that, when executed by the at least one processor, further cause the system to output a signal to the vaporizing device to activate a heating element disposed in the vaporizing device to convert the oil into a vapor for inhalation by the user.

18. The system of claim 16, the machine-readable instructions that, when executed by the at least one processor, further cause the system to create a user dosage for the oil based on the feedback from the user, wherein the user dosage is increased when the user feedback indicates low efficacy and decreased when the user feedback indicates excessive efficacy.

19. The system of claim 16, the machine-readable instructions that, when executed by the at least one processor, further cause the system to transmit a signal to the vaporizing device to record and output dosage data indicative of a duration of inhalation by the user when the user is administering the target dosage or a user dosage.

20. The system of claim 16, the machine-readable instructions that, when executed by the at least one processor, further cause the system to output a signal to the vaporizing device to block the user from administering the dosage.
